(19) United States
(12) Patent Application Publication
(10) Pub. No.: US 2025/0022316 A1
(43) Pub. Date: Jan. 16, 2025

(54) VISUAL HEALTH TRACKING SYSTEM

(71) Applicants: Rachit Girdhar, Piscataway, NJ (US); Sean Munjal, Ann Arbor, MI (US)

(72) Inventors: Rachit Girdhar, Piscataway, NJ (US); Sean Munjal, Ann Arbor, MI (US)

(21) Appl. No.: 18/350,300

(22) Filed: Jul. 11, 2023

Related U.S. Application Data
(60) Provisional application No. 63/388,041, filed on Jul. 11, 2022.

(51) Int. Cl. G06V 40/20; G06F 3/01; G06V 10/70; G06V 20/40; G16H 20/30

(52) U.S. Cl. CPC G06V 40/23; G06F 3/011; G06V 10/70; G06V 20/46; G16H 20/30

(57) ABSTRACT

A system for tracking and logging an athletic workout of a user, the system including at least one camera and at least one computing device running a tracking application. The system provides the user with tailored dialogue, logging, analysis and feedback based on the video, photo or audio information the camera receives from viewing the user and exercise equipment. The computing device includes a module for generating information via various artificial modalities by processing input data. The system may process visual inputs from the user, area, wearable or fitness-related item including their movement, equipment and utilizes machine learning models to analyze and extract relevant information.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### CROSS-REFERENCE TO RELATED APPLICATIONS

This application claims the benefit of U.S. Provisional Patent Application Ser. No. 63/388,041, filed on Jul. 11, 2022, which is incorporated by reference herein in its entirety.

### BACKGROUND OF THE INVENTION

### 1) Field of the Invention

The invention relates to a system for tracking and logging an athletic workout of a user and more specifically, to a system that utilizes artificial intelligence and machine learning to provide feedback to the user during and after a workout.

### 2) Description of Related Art

Currently to log a workout, some people jot down their progress using pen and paper and others use workout logging applications on their phone. The most common gym-workout applications are flawed because a user has to tap an excessively tedious number of times on a phone screen to log a single workout.

In particular there is a need for a solution to at least one of the aforementioned problems.

### BRIEF SUMMARY OF THE INVENTION

The invention minimizes the manual data entry drastically, by allowing the system to capture some, most or all of the loggable information.

The present invention is directed to a system for tracking and logging an athletic workout of a user, the system including at least one camera and at least one computing device running a tracking application. The system provides the user analysis and feedback based on the video information the camera receives from viewing the user and exercise equipment. The computing device includes a module for generating information by way of various artificial intelligent modalities by processing input data. The system may process visual inputs from the user, area, wearable or fitness-related item including their movement, equipment being used and the weight(s) attached, and utilizes machine learning models to analyze and extract relevant information. The at least one camera may be selected from the group consisting of but not limited to mobile device cameras, surveillance cameras, body cameras, web cameras, laptop cameras, action cameras, PTZ (Pan-Tilt-Zoom) cameras, IP cameras, thermal cameras, 360-degree cameras, drone cameras, infrared cameras, Digital Single-Lens Reflex cameras, mirrorless cameras, webcam cameras, network cameras, compact cameras, Single-Lens Translucent cameras, medium format cameras, Closed-Circuit Television cameras, bullet cameras, Dome cameras, underwater cameras, trail cameras, body-worn cameras and time-lapse cameras. The at least one camera may be placed in a location within the exercise equipment, within the user clothing or within a workout facility in which the user is performing the user athletic workout. The camera may be aimed at the user, the gym equipment, equipment attachment, wearable device, or any combination thereof. A select area may be developed by combining data including the target user's location within the fitness establishment with the help of mapping, GPS, facial recognition and data from sensors acquired through a wearable sensor worn by the user or the sensors installed in the facility. The wearable sensor may provide sufficient data to help enable the cameras to identify and focus on a specific user of interest. The system may provide an output of information, including the name of the gym equipment, attachment, fitness-related item, the name of the exercise, movement recognition, and any possible form feedback for the exercise being performed by the targeted user. The output may include exercise recommendations based on the analyzed data, user fitness level, user goals, and user preferences. The system may retrieve a list of exercises associated with the identified equipment from a local or cloud-based database, the exercises filtered based on factors such as muscle groups, duration, level of expertise, equipment type, and user-specific preferences. A user may interact with the system by selecting exercises through touch, verbal commands, or by initiating the exercise action. The system may provide options for customizing the exercise parameters, such as weight, sets, reps, duration, distance, speed, incline, steps, levels, floors and calories burned, based on the user's input. The system may allow users to log individual exercises into a workout session, which may be tracked from the start of the first logged exercise until the completion of the workout. When the workout session concludes, the system may log comprehensive data, including the list of exercises performed, time, duration, weight lifted, calories burned, and muscle groups worked on, which can be stored in a cloud-based platform and/or on the local device. The system may incorporate input from multiple sources of data of which the predominant source is cameras and may also include wearable sensors and sensory data in which the data is put into one or multiple deep learning models that may or may not be interacting with one another to provide users with an enhanced visual health tracking experience, minimizing manual data entry and providing real-time tailored dialogue, logging, analysis, feedback and recommendations based on the visual inputs acquired.

Another aspect of the present invention is directed to a system for tracking and logging a user athletic workout. The system includes a wearable sensor for targeted user identification that aids in identifying and targeting a specific user within a fitness establishment. The wearable sensor may provide sufficient data to enable the cameras to recognize and focus on the targeted user, the targeted user identification enhances the accuracy and effectiveness of the visual health tracking system. The wearable sensor may be a wristband, armband, smartwatch, fitness tracker, clothing, smart glasses, ear attachments, or any other wearable device capable of collecting and transmitting data. The system may incorporate sensors such as accelerometers, gyroscopes, magnetometers, location sensors, or other relevant sensors, to capture and transmit user movement and positional information. The visual health tracking system may determine the location of the user within the fitness establishment by utilizing mapping technology and the data acquired through the wearable sensor and the sensors installed at the facility. The system may establish a correlation between the captured visual inputs from the cameras and the data received from the user wearing the wearable sensor, allowing for accurate identification and tracking of the targeted user. The sensors that interact with the wearable sensor may continuously collect and transmit data to the system, providing information on the user's movement patterns, exercise actions, and equipment usage, the data then processed alongside the visual inputs from the cameras using the machine learning models within the system. The machine learning models may be trained to analyze and extract meaningful information from the combined data sources, including the visual inputs specific to the targeted user, enabling the system to provide personalized feedback, exercise recommendations, and real-time performance analysis to the user based on their captured movements, equipment usage, weights and other fitness-related items. The wearable sensor may encompass a wide range of options such as clothing, smart glasses, ear attachments, and other wearable devices, act as in creating a comprehensive and accurate visual health tracking experience for the system to focus on the targeted user and understand their specific activities within the fitness establishment, and deliver tailored information and insights based on their individualized visual input.

Another aspect of the present invention is directed to a system for using camera technology to visually track different areas of health. The system includes capturing an item of interest or person of interest by the camera being focused toward gym equipment, an attachment, a fitness-related item or an exercising individual and capturing an image or live view of the gym equipment, the attachment, the fitness-related item or the exercising individual and processing the individual image or each frame of a live feed. The system includes processing the input which may be processing an image or frame through a deep learning model, the deep learning model providing information such as, but not limited to, the name of the gym equipment, an amount of weight attached to said gym equipment and the name of an exercise, its movement recognition and any possible form feedback as it is being performed, as the output. The system includes getting the list of exercises including if the item of interest is a piece of gym equipment and/or attachment, the system searches the name of the item of interest received in the output through a database that may exist on a local device or cloud for the list of exercises associated with the received output. The system also includes an algorithm picking the best exercises for a user to do using the gym equipment, attachment or fitness-related item based on a plurality of factors. The system includes filtering the exercises including after the system has generated its output from the deep learning model, filtering the corresponding exercises of the identified gym equipment and/or attachment through different filters that may contain but are not limited to, muscle groups, duration, level of expertise, equipment type, or the like. The system includes selecting the exercise including once the person of interest has selected an exercise, by touch, verbal, or simply starting the action of the exercise, the person of interest may be given options based on the type of exercise, such as selecting the weight, number of sets, number of reps, duration, distance and calories burned. The system includes logging the exercise including after completing an exercise, the person of interest has the option to log that exercise into a workout session, the workout session begins when the person of interest logs their first exercise and ends when the person of interest completes their workout. The system may utilize location-based logging. Location-based logging includes logging information when the user enters into an area where the system cameras are located and logs the workout when person of interest exits that area. The system includes logging the workout including when a person of interest decides to end their workout, their entire workout session, can contain but isn't limited to the list of exercises, time, the duration, the weight lifted, calories burned, the distance covered and muscle groups worked on, is stored in a cloud and/or in the local device. The item of interest is a piece of gym equipment, attachment or fitness-related item and the person of interest is an exercising individual. The step of processing an input includes capturing a photo whereby a camera is pointed towards any gym equipment, attachment, fitness-related item or exercising individual and an image is captured. The step of processing an input includes a live feed whereby a camera is actively engaged towards any gym equipment, attachment, fitness-related item or exercising individual and each frame is processed.

These and other objects, features, and advantages of the present invention will become more readily apparent from the attached drawings and the detailed description of the preferred embodiments, which follow.

### BRIEF DESCRIPTION OF THE DRAWINGS

FIG. 1 shows some examples of devices containing cameras that may be used in the present invention;

FIG. 2 shows a pair of cameras set up to view various exercise equipment; and

FIG. 3 is a flowchart showing steps for using the visual health camera technology and system.

FIG. 4 shows a system for tracking and logging an athletic workout of a user according to the present invention.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

The terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The terms “person of interest” and “user” are used interchangeably to mean an individual who is either performing the exercise or using the system.

The invention is drawn to camera technology and a system for using the camera technology. The system may include the camera and any other equipment associated with digital video, storage of information captured by the video, the processing of the video and computer related hardware and software that carries out processing of the video information and processed information. The system allows for processing of video, photo and audio information captured the camera. The audio may alternately be captured by a microphone separate from the camera. The flowchart shows steps for using the visual workout tracking system and may be considered a method of use for the system as well as a method of use for using the camera and/or related equipment.

The instant invention is a system that allows at least one camera to help track gym workouts. If a person wanted to track a gym workout, manual data entry needed to be input to begin the process. Now, a system that utilizes at least one camera can recognize gym equipment, attachments, fitness-related items, or an exercising individual and provide specific information such as what an item is, what exercises or actions can be performed with it, the name of an exercise as it is being performed, how many repetitions have been performed, actual form feedback of an individual doing an exercise, with or without an item of interest.

The invention may be used by health-conscious individuals who are trying to track their fitness progress.

The invention has been created because prior to this, advanced computer vision models could not be scaled to small technology devices (i.e., smartphones, smart glasses)

Currently to log a workout, some people jot down their progress using pen and paper and others use workout logging applications on their phone. The most common gym-workout applications are flawed because a user has to tap an excessively tedious number of times on a phone screen to log a single workout. The invention minimizes the manual data entry, by allowing the system to capture a bulk of the loggable information.

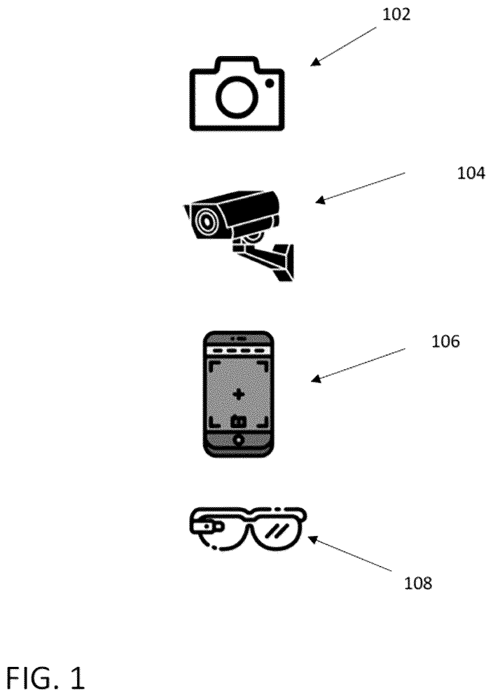

Referring now to the drawings FIGS. 1-5, and more particularly to FIG. 1, there is shown various cameras which may be implemented in the visual health tracking system. Cameras include but are not limited to photography cameras 102, surveillance type cameras 104, cell phone cameras 106, laptop cameras, tablet cameras, wearable cameras and eyeglass cameras 108.

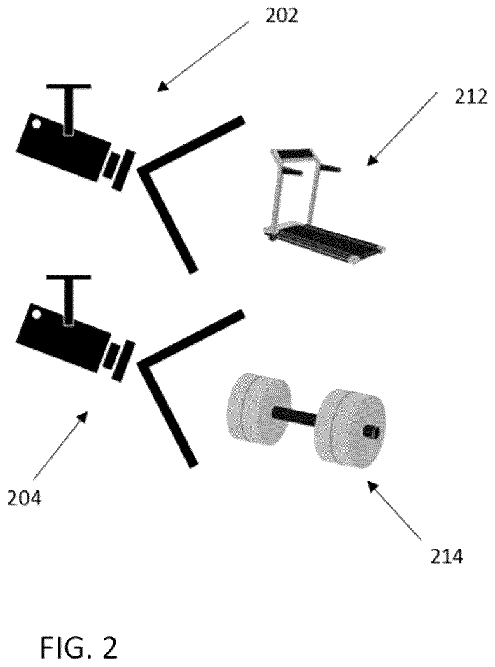

FIG. 2 shows a camera 202 aiming at a piece of gym equipment 212 and a camera 204 aiming at a second piece of gym equipment 214. This may represent the same camera aimed at different pieces of gym equipment at different times. Alternately, this may represent a plurality of cameras aiming at different gym equipment, or the same equipment. Besides gym equipment, this may represent cameras aimed at attachments, other fitness-related items, or exercising individuals.

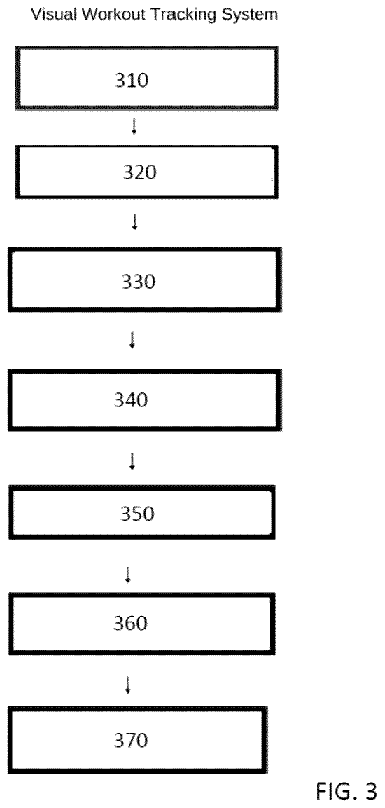

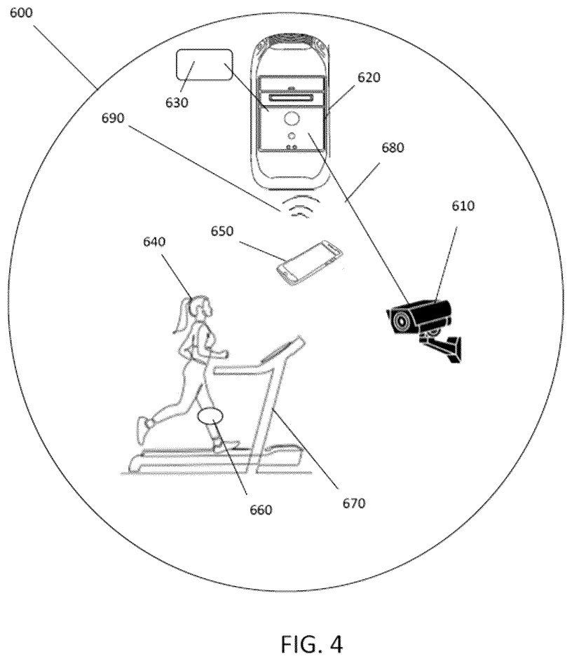

FIG. 3 shows a flowchart for using the system of the present invention. FIGS. 4 and 5 are an enlargement of FIG. 3 for clarity.

The steps include:
capturing an item of interest or person of interest 310;
processing the input 320;
getting the list of exercises 330;
filtering the exercises 340;
selecting the exercise 350;
logging the exercise 360; and
logging the workout 370.

Each of the steps are described below.

Capturing an item of interest or person of interest 310—One of these two methods may be used to capture the item of interest or person of interest:
a. Capturing a photo: A camera is aimed towards any gym equipment, attachment, fitness-related item or exercising individual and an image is captured.
b. Live Feed: A camera is actively engaged towards any gym equipment, attachment, fitness-related item or exercising individual and each frame is processed.

Processing the Input 320—Once the camera has captured a gym equipment, attachment, fitness-related item or exercising individual, then an image or frame is processed through our deep learning model. After processing that input, the deep learning model can provide information such as, but not limited to, the name of the gym equipment, the amount of weight attached and/or being lifted, and the exercise being performed, as the output.

Getting the list of exercises 330—If the item of interest is a gym equipment and/or attachment, the system searches the name of the item received in the output through a database that may exist on a local device or cloud for the list of exercises associated with that piece of gym equipment. An algorithm picks the best exercises for a user to do on that specific equipment based on a multitude of factors.

Filter the exercises 340—After the system has generated its output from the deep learning model, it can filter the corresponding exercises of the identified gym equipment and/or attachment through different filters that may contain but are not limited to, muscle groups, duration, level of expertise, equipment type, etc.

Selecting the exercise 350—Once the user has selected an exercise, through touch, verbal, or simply starting the action of the exercise, they may be given options based on the type of exercise, such as selecting the weight, number of sets, number of reps, duration, distance and calories burned.

Logging the exercise 360—After completing an exercise, the user has the option to log that exercise into a workout session. A workout session begins when the user logs their first exercise and ends when the user completes their workout.

Logging the workout 370—When a user decides to end their workout, their entire workout session, containing the list of exercises, time, the duration and muscle groups worked on, is stored in a cloud and/or in the local device.

Another embodiment of the present invention, as shown in FIG. 4, is directed to a system 600 for tracking and logging an athletic workout of a user 640, the system 600 including at least one camera 610 and at least one computing device 620 running a tracking application. The system provides the user 640 analysis and feedback based on the video information the camera receives and sends, by wire 680 or wireless system, from viewing the user 640 and exercise equipment 670. The computing device 620 includes an AI module 630 for generating AI information for processing input data. The system 600 processes visual inputs from the user 640, area, wearable device 660 or fitness-related item including their movement, equipment being used and the weight(s) attached, and utilizes machine learning models to analyze and extract relevant information. The at least one camera 610 may be selected from the group consisting of a mobile device camera, a surveillance camera and a body camera. The at least one camera 610 may be placed in a location within the exercise equipment 670, within the user clothing or within a workout facility in which the user 640 is performing the user athletic workout. The camera 610 may be aimed at the user 640, the gym equipment 670, equipment attachment, wearable device 660, or any combination thereof. A select area may be developed by combining data including the target user's location within the fitness establishment with the help of mapping, GPS, facial recognition and data from sensors acquired through a wearable sensor worn by the user. The wearable sensor 660 may provide sufficient data to help enable the cameras to identify and focus on a specific user of interest. The system 600 provides an output of information, including the name of the gym equipment, attachment, fitness-related item, the name of the exercise, movement recognition, and any possible form feedback for the exercise being performed by the targeted user. The output may include exercise recommendations based on the analyzed data, user fitness level, user goals, and user preferences. The system may retrieve a list of exercises associated with the identified equipment from a local or cloud-based database, the exercises filtered based on factors such as muscle groups, duration, level of expertise, equipment type, and user-specific preferences. A user may interact with the system by selecting exercises through touch, verbal commands, or by initiating the exercise action. The system may provide options for customizing the exercise parameters, such as weight, sets, reps, duration, distance, and calories burned, based on the user's input. The system may allow users to log individual exercises into a workout session, which is tracked from the start of the first logged exercise until the completion of the workout. When the workout session concludes, the system may log comprehensive data, including the list of exercises performed, time, duration, weight lifted, calories burned, and muscle groups worked on, which can be stored in a cloud-based platform and/or on the local device. The system may incorporate input from multiple sources of data of which the predominant source is cameras and may also include wearable sensors and sensory data in which the data is put into one or multiple deep learning models that may or may not be interacting with one another to provide users with an enhanced visual health tracking experience, minimizing manual data entry and providing real-time feedback and recommendations based on the visual inputs acquired. The camera provides video information and may also provide audio information used by the system. The system may also use a plurality of cameras while employing the same or different cameras within the same system. The one camera or the plurality of cameras are selected from the group consisting of but not limited to mobile device cameras, surveillance cameras, body cameras, web cameras, laptop cameras, action cameras, PTZ (Pan-Tilt-Zoom) cameras, IP cameras, thermal cameras, 360-degree cameras, drone cameras, infrared cameras, Digital Single-Lens Reflex cameras, mirrorless cameras, webcam cameras, network cameras, compact cameras, Single-Lens Translucent cameras, medium format cameras, Closed-Circuit Television cameras, bullet cameras, Dome cameras, underwater cameras, trail cameras, body-worn cameras and time-lapse cameras.

Also shown in FIG. 4 is another aspect of the present invention directed to a system for tracking and logging a user athletic workout. The system 600 includes a wearable sensor 660 for targeted user identification that aids in identifying and targeting a specific user 640 within a fitness establishment. The wearable sensor 660 provides sufficient data to enable the cameras to recognize and focus on the targeted user 640, the targeted user identification enhances the accuracy and effectiveness of the visual health tracking system. The wearable sensor 660 may be a wristband, armband, smartwatch, fitness tracker, clothing, smart glasses, ear attachments, or any other wearable device capable of collecting and transmitting data. The system 600 incorporates sensors such as accelerometers, gyroscopes, magnetometers, location sensors, or other relevant sensors, to capture and transmit user movement and positional information. The sensors may communicate by hardwire, Bluetooth, Wi-Fi, ZigBee or any other wireless communication. The sensors may incorporate Bluetooth Low Energy beacons or other beacons. The visual health tracking system may determine the location of the user within the fitness establishment by utilizing mapping technology and the data acquired through the wearable sensor. The system 600 establishes a correlation between the captured visual inputs from the cameras 610 and the data received from the user 640 wearing the wearable sensor 660, allowing for accurate identification and tracking of the targeted user 640. The sensors that interact with the wearable sensor continuously collects and transmits data to the system computing device 620, providing information on the user's movement patterns, exercise actions, and equipment usage, the data then processed alongside the visual inputs from the cameras using the machine learning models within the system. The machine learning models 630 are trained to analyze and extract meaningful information from the combined data sources, including the visual inputs specific to the targeted user, enabling the system to provide personalized feedback, exercise recommendations, and real-time performance analysis to the user based on their captured movements, equipment usage, weights and other fitness-related items. The wearable sensor 660 may encompass a wide range of options such as clothing, smart glasses, ear attachments, and other wearable devices, act as in creating a comprehensive and accurate visual health tracking experience for the system to focus on the targeted user and understand their specific activities within the fitness establishment, and deliver tailored information and insights based on their individualized visual input.

In some embodiments the method or methods described above may be executed or carried out by a computing system including a tangible computer-readable storage medium or storage including but not limited to a cloud or a remotely hosted database, also described herein as a storage machine, which holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, DVD or cloud-based storage devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI), or any visual element of the methods or processes described above. For example, the display subsystem, storage machine, and logic machine may be integrated such that the above method may be executed while visual elements of the disclosed system and/or method are displayed on a display screen or a holographic display for user consumption. The computing system may include an input subsystem that receives user input. The input subsystem may be configured to connect to and receive input from devices including but not limited to a mouse, keyboard, gaming controller, mobile device, or any device that has a camera. For example, a user input may indicate a request that certain task is to be executed by the computing system, such as requesting the computing system to display any of the above described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API) or other server-client based communication protocols.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A system for tracking and logging an athletic workout of a user, the system comprising:
at least one camera; and
at least one computing device running a tracking application;
wherein the system provides the user analysis and feedback based on video, photo or audio information the camera receives from viewing the user and exercise equipment;
wherein the system provides the user with tailored dialogue, logging, analysis, feedback and recommendations; and
wherein the computing device includes a module for generating information via various artificial intelligence modalities by processing input data.

2. The system according to claim 1 wherein the system processes visual inputs from the user, area, wearable or fitness-related item including their movement, equipment being used, the weight(s) attached, location sensors, Bluetooth sensors and/or wireless sensor, and utilizes machine learning models to analyze and extract relevant information.

3. The system according to claim 1 wherein the at least one camera is selected from the group consisting of:
mobile device cameras;
surveillance cameras;
body cameras;
web cameras;
laptop cameras;
action cameras;
PTZ (Pan-Tilt-Zoom) cameras;
IP cameras;
thermal cameras;
360-degree cameras;
drone cameras;
infrared cameras;
Digital Single-Lens Reflex cameras;
mirrorless cameras;
webcam cameras;
network cameras;
compact cameras;
Single-Lens Translucent cameras;
medium format cameras;
Closed-Circuit Television cameras;
bullet cameras;
Dome cameras;
underwater cameras;
trail cameras;
body-worn cameras; and
time-lapse cameras.

4. The system according to claim 1 wherein the at least one camera is placed in a location selected from the group consisting of:
within the exercise equipment;
within the user clothing; and
within a workout facility in which the user is performing the user athletic workout.

5. The system according to claim 1 wherein the camera is aimed at the user, the gym equipment, equipment attachment, wearable device, or any combination thereof.

6. The system according to claim 1 wherein a select area is developed by combining data including the target user's location within the fitness establishment with the help of mapping, GPS, facial recognition and data from sensors acquired through a wearable sensor worn by the user.

7. The system according to claim 6 wherein the wearable sensor may provide sufficient data to help enable the cameras to identify and focus on a specific user of interest.

8. The system according to claim 1 wherein the system provides an output of information, including the name of the gym equipment, attachment, fitness-related item, the name of the exercise, movement recognition, and any possible form feedback for the exercise being performed by the targeted user.

9. The system according to claim 1 wherein the output includes exercise recommendations based on the analyzed data, user fitness level, user goals, and user preferences.

10. The system according to claim 1 the system retrieves a list of exercises associated with the identified equipment from a local or cloud-based database, the exercises filtered based on factors such as muscle groups, duration, level of expertise, equipment type, and user-specific preferences.

11. The system according to claim 1 wherein a user interacts with the system by selecting exercises through touch, verbal commands, or by initiating the exercise action. The system provides options for customizing the exercise parameters, such as weight, sets, reps, duration, distance, speed, incline, steps, levels, floors and calories burned, based on the user's input. The system allows users to log individual exercises into a workout session, which is tracked from the start of the first logged exercise until the completion of the workout. When the workout session concludes, the system logs comprehensive data, including the list of exercises performed, time, duration, weight lifted, calories burned, and muscle groups worked on, which can be stored in a cloud-based platform and/or on the local device.

12. The system according to claim 1 wherein the system incorporates input from multiple sources of data of which the predominant source is cameras and may also include wearable sensors and sensory data in which the data is put into one or multiple deep learning models that may or may not be interacting with one another to provide users with an enhanced visual health tracking experience, minimizing manual data entry and providing real-time feedback and recommendations based on the visual inputs acquired.

13. A system for tracking and logging a user athletic workout, the system comprising:
a wearable sensor for targeted user identification that aids in identifying and targeting a specific user within a fitness establishment.

14. The system according to claim 13 wherein the wearable sensor is designed to provide sufficient data to enable the cameras to recognize and focus on the targeted user, the targeted user identification enhances the accuracy and effectiveness of the visual health tracking system.

15. The system according to claim 13 wherein the wearable sensor can take various forms including wristbands, armbands, smartwatches, fitness trackers, clothing, smart glasses, ear attachments, or any other wearable device capable of collecting and transmitting data.

16. The system according to claim 13 wherein the system incorporates sensors such as accelerometers, gyroscopes, magnetometers, location sensors, or other relevant sensors, to capture and transmit user movement and positional information.

17. The system according to claim 13 wherein the visual health tracking system determines the location of the user within the fitness establishment by utilizing mapping technology and the data acquired through the wearable sensor.

18. The system according to claim 13 wherein the sensors that interact with the wearable sensor continuously collect and transmit data to the system, providing information on the user's movement patterns, exercise actions, and equipment usage, the data then processed alongside the visual inputs from the cameras using the machine learning models within the system.

19. The system according to claim 13 wherein the machine learning models are trained to analyze and extract meaningful information from the combined data sources, including the visual inputs specific to the targeted user, enabling the system to provide personalized feedback, exercise recommendations, and real-time performance analysis to the user based on their captured movements, equipment usage, weights and other fitness-related items.

20. A visual machine learning system for tracking and logging a user athletic workout, the system comprising:
pointing a camera towards a piece of gym equipment, an attachment, a fitness-related item or an exercising individual and capturing an image or live feeding from the camera actively engaged towards gym equipment, an attachment, a fitness-related item or an exercising individual and processing a photo or each frame of the live feed to capture an item of interest or person;
processing the input including processing one of the image or frame through a deep learning model, the deep learning model providing information such as, but not limited to, the name of the gym equipment, an amount of weight attached to said gym equipment, and the name of an exercise, its movement recognition, and any possible form feedback on the exercise being performed, as the output;
getting the list of exercises including if the item of interest is a piece gym equipment and/or attachment, the system searches the name of the item of interest received in the output through a database that may exist on a local device or cloud for the list of exercises associated with the piece of gym equipment and an algorithm picking the best exercises for a user to do on the piece of gym equipment based on a plurality of factors;
filtering the exercises including after the system has generated its output from the deep learning model, filtering the corresponding exercises of the identified gym equipment and/or attachment through different filters that may contain but are not limited to, muscle groups, duration, level of expertise, equipment type, or the like;
selecting the exercise including once the person of interest has selected an exercise, by touch, verbal, or simply starting the action of the exercise, the person of interest may be given options based on the type of exercise, such as selecting the weight, number of sets, number of reps, duration, distance and calories burned;
logging the exercise including after completing an exercise, the person of interest has the option to log that exercise into a workout session, the workout session begins when the person of interest logs their first exercise and ends when the person of interest completes their workout; and
logging the workout including when a person of interest decides to end their workout, their entire workout session, containing the list of exercises, time, the duration and muscle groups worked on, is stored in a cloud and/or in the local device;
wherein the item of interest is a piece of gym equipment, attachment or fitness-related item and the person of interest is an exercising individual;
wherein the step of processing an input includes capturing a photo whereby a camera is pointed towards any gym equipment, attachment, fitness-related item or exercising individual and an image is captured; and
wherein the step of processing an input includes a live feed whereby a camera is actively engaged towards any gym equipment, attachment, fitness-related item or exercising individual and each frame is processed.
